(19) United States
(12) Patent Application Publication
(10) Pub. No.: US 2023/0090684 A1
(43) Pub. Date: Mar. 23, 2023

(54) A Modular Catheter System

(71) Applicant: The Board of Trustees of the Leland Stanford Junior University, Stanford, CA (US)

(72) Inventors: Avnesh S. Thakor, Palo Alto, CA (US); Annabel Imbrie-Moore, Redwood City, CA (US)

(21) Appl. No.: 17/904,051

(22) PCT Filed: Feb. 10, 2021
(86) PCT No.: PCT/US2021/017468

§ 371 Date: Aug. 11, 2022

Related U.S. Application Data
(60) Provisional application No. 62/975,407, filed on Feb. 12, 2020.

(51) Int. Cl. A61M 25/00; A61M 1/36; A61M 1/28

(52) U.S. Cl. CPC A61M 25/0026; A61M 25/0097; A61M 25/0068; A61M 1/3661; A61M 1/285; A61M 2209/088; A61M 2240/00

(57) ABSTRACT

A modular tunneled catheter is provided that allows lumen length and cuff positioning to be adjusted based on the size of a patient. This catheter should be useful for any patient who would benefit from customized fitting of a catheter to their measurements. Methods of using the modular catheter for hemodialysis, peritoneal dialysis, and fluid drainage, including drainage of plural effusions from the lungs or malignant ascites from the abdomen are also described.

### BACKGROUND OF THE INVENTION

Pediatric patients with acute kidney injury and end stage renal disease continue to require hemodialysis, yet the available central venous catheters are generally sized for adults. As a result, children are subject to unnecessary patient discomfort with catheters having excessive subcutaneous tract lengths, incorrect internal tip positions, and excessive catheter diameters, which contribute to risks of arrhythmia, thrombosis, and poor dialysis. Physicians face challenges when placing dialysis catheters when devices are too large for children. The limited options make optimal sizing difficult or even impossible in some cases.

Thus, there remains a need for the development of a catheter system with components having suitable dimensions and positioned more appropriately for the smaller sized pediatric patient.

### SUMMARY OF THE INVENTION

A modular tunneled catheter is provided that allows lumen length and cuff positioning to be adjusted based on the size of a patient. This modular catheter overcomes problems with the use of other tunneled catheters currently available on the market that poorly fit infants, adolescents, and adults of uncommon sizes and should be useful for any patient who would benefit from customized fitting of a catheter to their measurements. The modular catheters described herein can be used in various applications such as hemodialysis, peritoneal dialysis, and fluid drainage, including drainage of plural effusions from the lungs or malignant ascites from the abdomen.

In one aspect, an adjustable cuff is provided comprising: a) a cuff body comprising a cylindrical surface with a slit, said cuff body having grooved edges on either side of the slit, wherein the cuff body fits around a catheter lumen and is slidable along the catheter lumen to allow custom positioning of the adjustable cuff along the catheter lumen when the cuff is unlocked; and b) a cuff key comprising projections that fit into the grooved edges at a complementary receiving location for the cuff key, wherein inserting the cuff key into the grooved edges at the receiving location locks the cuff and secures the cuff to the catheter lumen such that the cuff is no longer slidable along the catheter lumen. In certain embodiments, the adjustable cuff further comprises a layer of adhesive (e.g., biocompatible silicone) on the inner surface (i.e., lumen-facing surface when attached to a catheter) of the cuff body. In some embodiments, the inner surface of the cuff body further comprises a depression comprising a complementary receiving location for the adhesive.

In another aspect, an adjustable cuff is provided comprising a clip body that can assume an open position or a closed position, said clip body further comprising a cuff material on the surface of the clip body with an adhesive-compatible surface, wherein the clip body comprises a first cuff piece and a second cuff piece, each connected to a central hinge, wherein the first cuff piece and the second cuff piece can snap together around a catheter lumen to allow custom positioning of the adjustable cuff along the catheter lumen.

In certain embodiments, the clip body further comprises an external latching projection on the first cuff piece and a complementary latch receiving location on the second cuff piece, wherein the external latching projection of the first cuff piece fits inside the receiving location of the second cuff piece to latch closed the clip body when the clip body assumes the closed position.

In certain embodiments, the clip body further comprises an adhesive on the adhesive-compatible surface of the cuff material. In some embodiments, the adhesive is an activatable adhesive.

In another aspect, a modular catheter is provided comprising: a) a hub comprising a pair of access ports, wherein the pair of access ports comprises a first access port for a fluid exiting a body of a patient and a second access port for a fluid entering the body of the patient; b) a trimmable lumen that can be trimmed to a customizable length, wherein the trimmable lumen comprises: i) a first end that is trimmable, wherein the first end is attached to a hub connector and a sealing ring that are used to connect the first end of the trimmable lumen to the hub by push-together assembly, and ii) a second end comprising a flow tip comprising a plurality of venous inlets and arterial outlets; and c) an adjustable cuff described herein.

In certain embodiments, the adjustable cuff is positioned on the trimmable lumen such that the length of a first section of the trimmable lumen between the flow tip and the modular venous-arterial hub and the length of a second section of the trimmable lumen between the flow tip and the adjustable cuff are customized to optimize tip spacing for a patient.

In certain embodiments, the length of the lumen and the position of the adjustable cuff are customized for antegrade tunneling or retrograde tunneling.

In certain embodiments, the length of the lumen and the position of the adjustable cuff are customized to allow placement of the lumen in a major vein of the patient, including, without limitation, an internal jugular vein, a subclavian vein, and a femoral vein. For example, if the major vein is the right internal jugular vein, the length of the lumen and the position of the adjustable cuff are customized to allow placement of the flow tip in the right atrium. If the major vein is the left internal jugular vein, the length of the lumen and the position of the adjustable cuff are customized to allow placement of the flow tip in the left atrium. If the major vein is the subclavian vein, the length of the lumen and the position of the adjustable cuff are customized to allow placement of the flow tip in the superior vena cava. If the major vein is the femoral vein, the length of the lumen and the position of the adjustable cuff are customized to allow placement of the flow tip in the inferior vena cava.

In certain embodiments, the catheter is sized for a pediatric patient such as an adolescent or an infant.

In certain embodiments, the trimmable lumen comprises polyurethane or silicone.

In certain embodiments, the trimmable lumen is a single lumen, a double lumen, or a triple lumen.

In some embodiments, the flow tip is a step-tip, split-tip, or symmetrical tip.

In certain embodiments, the length (distance from the hub to the flow tip) of the modular catheter ranges from about 2 cm to about 22 cm, from about 2 cm to about 12 cm, from about 13 cm to about 20 cm, or any length within these ranges such as 2, 3, 4, 5, 6, 7, 8, 9, 10, 11, 12, 13, 14, 15, 16, 17, 18, 19, 20, 21, or 22 cm.

In certain embodiments, the trimmable lumen has a French size ranging from 3 french to 34 french, from 10 french to 16 french, or from 3 french to 8 french, or any size within these ranges such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 french.

In certain embodiments, the modular catheter further comprises a catheter cuff positioning ratchet, wherein the ratchet comprises a plurality of cuff mounting projections that can engage the cuff. In some embodiments, the cuff further comprises a complementary ratchet receiving location designed to mate with at least one cuff mounting projection on the ratchet such that the cuff can be locked in place at the cuff mounting projection. In certain embodiments, the cuff positioning ratchet is attached to the catheter such that the cuff can be positioned along the lumen at a selected cuff mounting projection along the ratchet.

In another aspect, a hemodialysis system is provided comprising a modular catheter, described herein, and a dialyzer. In certain embodiments, the hemodialysis system further comprises a blood pressure monitor. In certain embodiments, the hemodialysis system further comprises a pump. In certain embodiments, the hemodialysis system further comprises a dialysis machine.

In another aspect, a peritoneal dialysis system is provided comprising a modular catheter, described herein, and a cycler machine.

In another aspect, a fluid drainage system is provided comprising a modular catheter, described herein, and a drainage container for collecting fluid. In certain embodiments, the fluid that is drained by the fluid drainage system comprises plural effusions drained from the chest or malignant ascites drained from the abdomen of a patient.

In another aspect, a method of using a modular catheter to perform hemodialysis on a patient is provided, the method comprising: a) taking measurements of the patient at a site of interest for catheter implantation; b) trimming the first end of the trimmable lumen and positioning and closing the adjustable cuff on the trimmable lumen of the modular catheter to customize the lumen length and flow tip spacing according to the measurements of the patient; c) attaching the hub connector and the sealing ring to the first end of the trimmable lumen; d) connecting the hub connector to the hub; e) implanting the modular catheter into a subcutaneous tunnel at the site of interest; f) placing the trimmable lumen within a major vein of the patient; g) positioning the flow tip in a suitable location based on said placing of the trimmable lumen within the major vein; h) connecting the pair of access ports of the hub to a dialysis machine, wherein the first access port is used for blood leaving the patient's body and the second access port is used for blood returning to the patient's body after being processed by the dialysis machine; and i) performing said hemodialysis on the patient.

In certain embodiments, the major vein is selected from the group consisting of an internal jugular vein, a subclavian vein, and a femoral vein. For example, if the major vein is the right internal jugular vein, the flow tip is positioned in the right atrium. If the major vein is the left internal jugular vein, the flow tip is positioned in the left atrium. If the major vein is the subclavian vein, the flow tip is positioned in the superior vena cava. If the major vein is the femoral vein, the flow tip is positioned in the inferior vena cava.

In certain embodiments, the method further comprises tunneling a portion of the modular catheter beneath the skin of the patient, for example, using antegrade tunneling or retrograde tunneling.

In certain embodiments, the method further comprises monitoring blood pressure of the patient while performing hemodialysis.

In certain embodiments, the method further comprises applying an adhesive on the adhesive-compatible surface of the cuff material on the surface of the clip body to adhere the adjustable cuff to the trimmable lumen when the clip body is in the closed position around the trimmable lumen. In some embodiments, the method further comprises activating the adhesive if the adhesive is an activatable adhesive.

In another aspect, a method of using a modular catheter to perform peritoneal dialysis on a patient is provided, the method comprising: a) taking measurements of the patient at a site of interest for catheter implantation in the patient's abdomen; b) trimming the first end of the trimmable lumen and positioning and closing the adjustable cuff on the trimmable lumen of the modular catheter to customize the lumen length and flow tip spacing according to the measurements of the patient; c) attaching the hub connector and the sealing ring to the first end of the trimmable lumen; d) connecting the hub connector to the hub; e) implanting the modular catheter at the site of interest in the abdomen; f) placing the trimmable lumen within a subcutaneous tunnel at the site of interest in the abdomen of the patient; g) positioning the flow tip within the abdominal cavity; h) connecting the pair of access ports of the hub to a cycler machine, wherein the first access port is used for draining fluid from the abdomen and the second access port is used for filling the abdomen with dialysate; and i) performing said peritoneal dialysis on the patient.

In certain embodiments, the method further comprises tunneling a portion of the modular catheter beneath the skin of the patient, for example, using antegrade tunneling or retrograde tunneling.

In certain embodiments, the method further comprises applying an adhesive on the adhesive-compatible surface of the cuff material on the surface of the clip body to adhere the adjustable cuff to the trimmable lumen when the clip body is in the closed position around the trimmable lumen. In some embodiments, the method further comprises activating the adhesive if the adhesive is an activatable adhesive.

In another aspect, a method of using a modular catheter to drain fluid from a patient is provided, the method comprising: a) taking measurements of the patient at site of interest for catheter implantation to allow fluid drainage; b) trimming the first end of the trimmable lumen and positioning and closing the adjustable cuff on the trimmable lumen of the modular catheter to customize the lumen length and flow tip spacing according to the measurements of the patient; c) attaching the hub connector and the sealing ring to the first end of the trimmable lumen; d) connecting the hub connector to the hub; e) implanting the modular catheter at the site of interest; f) placing the trimmable lumen within a subcutaneous tunnel at the site of interest in the patient; g) positioning the flow tip within the site of interest to allow fluid drainage; h) connecting at least one access port of the hub to a drainage line on a drainage container; i) collecting the fluid in the drainage container.

In certain embodiments, the site of interest for catheter implantation is in the chest or abdomen of the patient.

In certain embodiments, the fluid comprises plural effusions drained from the chest or malignant ascites drained from the abdomen of the patient.

In certain embodiments, the method further comprises tunneling a portion of the modular catheter beneath the skin of the patient, for example, using antegrade tunneling or retrograde tunneling.

In certain embodiments, the method further comprises applying an adhesive on the adhesive-compatible surface of the cuff material on the surface of the clip body to adhere the adjustable cuff to the trimmable lumen when the clip body is in the closed position around the trimmable lumen. In some embodiments, the method further comprises activating the adhesive if the adhesive is an activatable adhesive.

In the practice of any of the subject methods, the modular catheter may be further sutured securely in place in the body of the patient.

In certain embodiments, an anti-coagulant, an antibiotic, an analgesic agent, or a combination thereof is administered to the patient prior to, during, or after implantation of the modular catheter in the patient.

In another aspect, a kit comprising a modular catheter described herein is provided. In certain embodiments, the kit further comprises instructions for using the modular catheter for hemodialysis, peritoneal dialysis, or draining fluid from a patient. In some embodiments, the modular catheter is assembled and contained in a sterile package. In other embodiments, the modular catheter is unassembled and the hub, the trimmable lumen, and the adjustable cuff are contained in one or more sterile packages.

### BRIEF DESCRIPTION OF THE DRAWINGS

FIG. 1 shows a schematic of a modular catheter featuring a trimmable lumen to provide a customizable lumen length and an adjustable snap-together cuff with an option for applying adhesives that permits custom positioning of the cuff along the lumen. This modular catheter supports antegrade or retrograde tunneling and can be customized, in particular, for pediatric patients (supports pediatric flowrates and higher heart rates) as well as adults who could benefit from customized fitting of a catheter to their measurements.

FIG. 2 shows a schematic depicting how the modular catheter is assembled.

FIG. 3 shows pediatric modular catheter patient customization for an adolescent (left) and an infant (right). The modular catheter allows the Cuff-to-Tip (x₁) and Tip-to-Hub (y₁) distances to be customized based on measurements of a patient.

FIG. 4 shows advantages of the modular catheter for physicians, patients, and providers.

FIG. 5 shows a schematic of a modular pediatric catheter for hemodialysis that supports: customizable tip spacing (cut at #1), customizable length (cut at #2), and pediatric flowrates and BPM's.

FIG. 6 shows the procedure for customization of the modular catheter for a patient. The overall length needed for the catheter and the tip-to-tip lengths are determined. The lumen (hub not attached) and distal tip are cut to a desired length prior to assembly. The catheter may be placed for hemodialysis with either antegrade or retrograde tunneling. After the catheter system is secured and sutured, hemodialysis can begin.

FIG. 7 shows hub assembly. The cuff is placed onto the catheter and is secured at a desired location. The modular hub is pushed into each lumen at the cut end of the catheter. A crimp sleeve is engaged to secure the catheter to the hub before the outer sleeve is screwed into place.

FIG. 8 shows computational fluid dynamics (CDF) modeling for a short split-tip catheter having holes only 3 cm at the catheter tip. CDF modeling indicated that there is no blood re-circulation with this design.

FIG. 9 shows an initial design of the modular cuff. This design comprises a single body with a slit along the longitudinal axis that enables smooth motion of the cuff along the catheter. The edges of the slit are designed to serve as a clamp: when the cuff is squeezed, the clamp engages, and the cuff became a seamless cylinder secured to the catheter with significant holding force.

FIGS. 10A-10C show an advanced design of the modular cuff with an insert. This design comprises two parts: a main body with a longitudinal slit featuring grooves, and a smaller key with features that slot into the grooves (FIGS. 10A-10C). FIG. 10A shows a view of the cuff with the key uninserted and FIGS. 10B-10C show views of the cuff with the key partially inserted and fully inserted, respectively. When the key is slotted into the main cuff body, the cuff clamps down on the catheter, securing it in position. The main body additionally includes an inner layer of biocompatible silicone (SIL30, Carbon) that is 3D-printed to precisely fit into a matching depression on the inner face of the cuff main body. This silicone component significantly increases the friction between the catheter and cuff when clamped, thus resulting in a higher holding force.

FIGS. 11A-11B show a 3D printed modular cuff connected to catheter tip material with the key either partially inserted (FIG. 11A) or fully inserted (FIG. 11B) into the cuff.

### DETAILED DESCRIPTION OF THE INVENTION

A modular tunneled catheter is provided that allows lumen length and cuff positioning to be adjusted based on the size of a patient. This catheter should be useful for any patient who would benefit from customized fitting of a catheter to their measurements. Methods of using the modular catheter for hemodialysis, peritoneal dialysis, and fluid drainage, including drainage of plural effusions from the lungs or malignant ascites from the abdomen are also described.

Before the present compositions, methods, and kits are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a catheter” includes a plurality of such catheters and reference to “the adhesive” includes reference to one or more adhesives and equivalents thereof, e.g. glues, pastes, sealants, or activatable adhesives known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

In the description that follows, a number of terms conventionally used in the field are utilized. In order to provide a clear and consistent understanding of the specification and claims, and the scope to be given to such terms, the following definitions are provided. As will be appreciated by one of ordinary skill in the art, the present disclosure pertains to catheters, shunts, drainage tubes and the like generally referred to herein as “catheters”. The term “catheter” encompasses both long-term and short-term tunneled catheters, including those used for performing hemodialysis (e.g., accessing an internal jugular vein, a subclavian vein, or a femoral vein), peritoneal dialysis (e.g., accessing the abdomen), or fluid drainage (e.g., removal of fluid from the lungs, abdomen, liver, abscesses, cysts, pseudocysts, pneumothoraces, hematomas, bilomas, or urinomas). In some instances, catheters are tunneled through a subcutaneous tunnel in the chest or abdomen of a patient. However, it is not intended that the present disclosure be limited to such. It will also be appreciated that the term “catheter” can also apply to tunneled catheters positioned in other locations in the body of a patient. The term “catheter” may include, without limitation, dialysis catheters, drainage catheters, short-term or long-term tunneled venous catheters, high-flow catheters, high-pressure catheters, thin-walled catheters, and peripherally inserted central catheters (PICC).

The term “about,” particularly in reference to a given quantity, is meant to encompass deviations of plus or minus five percent.

The terms “recipient”, “individual”, “subject”, “host”, and “patient”, are used interchangeably herein and refer to any mammalian subject for whom treatment or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

A “therapeutically effective dose” or “therapeutic dose” is an amount sufficient to effect desired clinical results (i.e., achieve therapeutic efficacy). A therapeutically effective dose can be administered in one or more administrations.

Modular Catheter with a Trimmable Lumen and an Adjustable Cuff

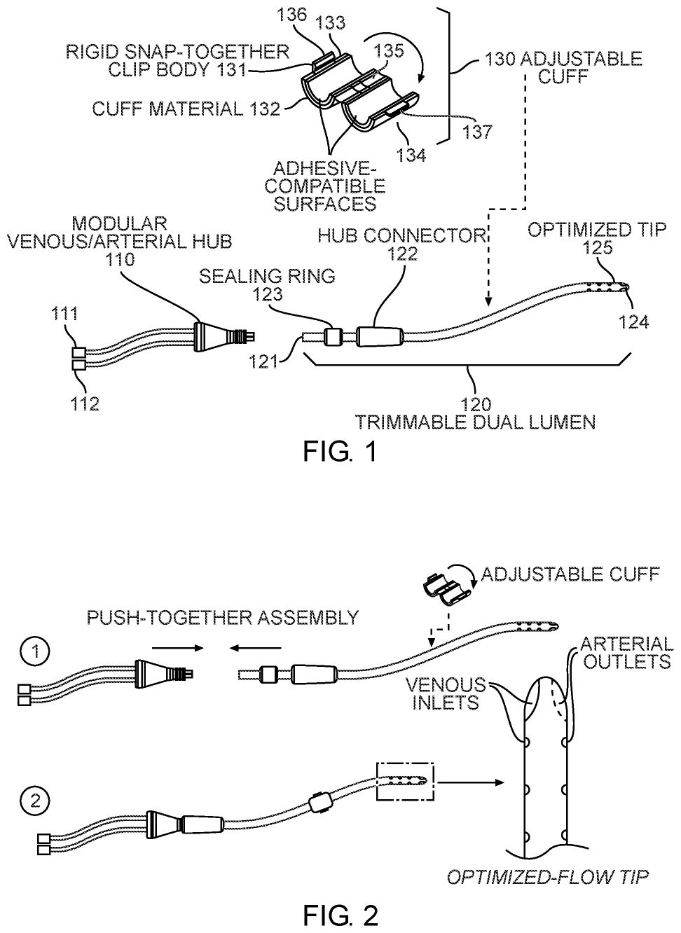

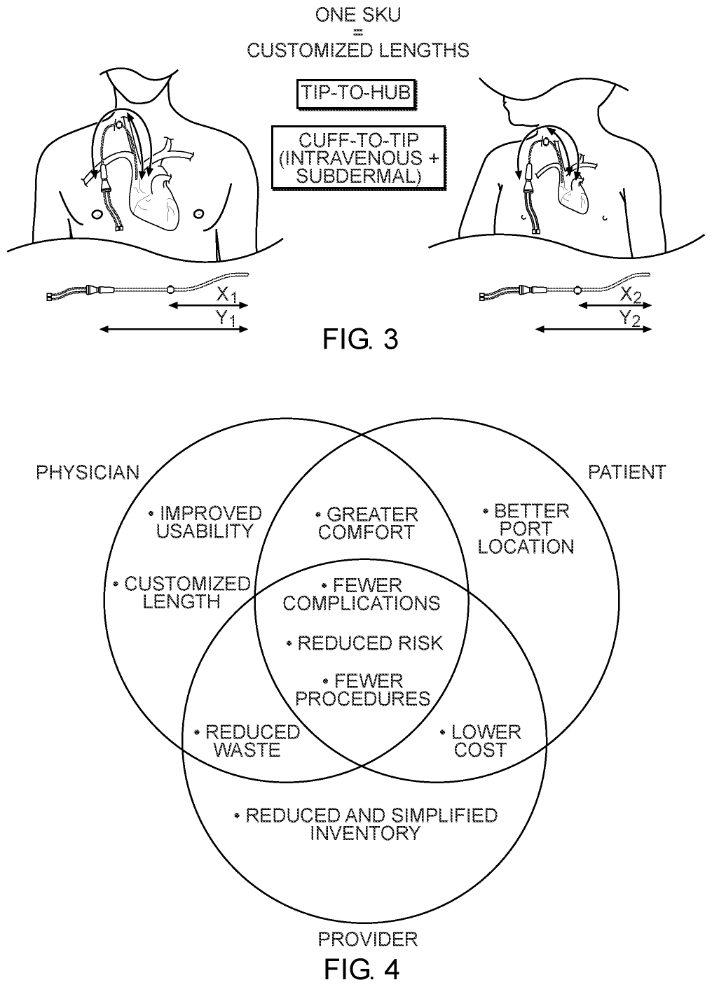

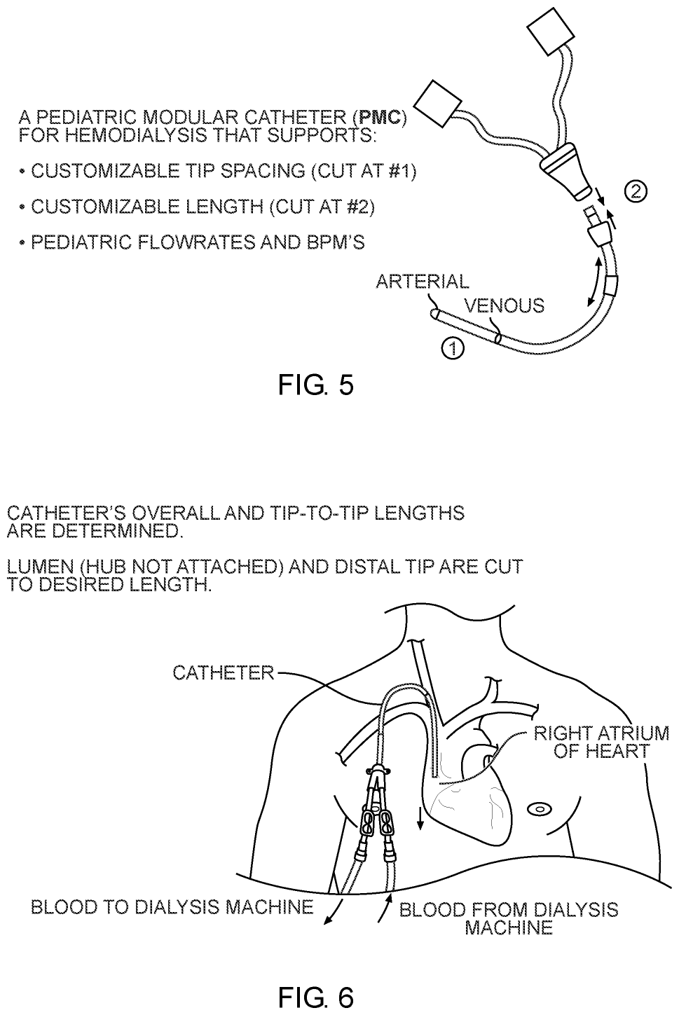

The present disclosure pertains to a modular catheter with a trimmable lumen and an adjustable cuff. As shown in FIG. 1, an exemplary modular catheter 100 comprises: a) a hub 110 comprising a pair of access ports, wherein the pair of access ports comprises a first access port 111 for fluid exiting the body of a patient and a second access port 112 for fluid entering the body of a patient; b) a trimmable lumen 120 that can be trimmed to a customizable length, wherein the trimmable lumen comprises: i) a first end that is trimmable 121, wherein the first end is attached to a hub connector 122 and a sealing ring 123 that are used to connect the first end 121 of the trimmable lumen to the hub 110 by push-together assembly, and ii) a second end 124 comprising a flow tip 125 comprising a plurality of venous inlets and arterial outlets; and c) an adjustable cuff 130 (or 230). The adjustable cuff can be placed at a selected position on the lumen either before or during surgery. After implantation of the catheter, the patient's subcutaneous tissue is allowed to grow into the cuff, thus securing the catheter and reducing the risk of infection.

In certain embodiments, the adjustable cuff 130 comprises a clip body 131 that can assume an open position or a closed position. The clip body 131 comprises a first cuff piece 133 and a second cuff piece 134, each connected to a central hinge 135, wherein the first cuff piece and the second cuff piece can snap together around a trimmable lumen to allow custom positioning of the adjustable cuff along the trimmable lumen. The clip body may further comprise a cuff material 132 on the surface of the clip body with an adhesive-compatible surface. In some embodiments, the clip body 131 further comprises an external latching projection 136 on the first cuff piece 133 and a complementary latch receiving location 137 on the second cuff piece 134, wherein the external latching projection of the first cuff piece fits inside the receiving location of the second cuff piece to latch closed the clip body when the clip body assumes the closed position. The cuff clip body can be closed by mechanically snapping together the two cuff pieces around the lumen. In some embodiments, the clip body comprises an external latching projection on the first cuff piece that mates with a complementary latch receiving location on the second cuff piece such that the external latching projection of the first cuff piece fits inside the receiving location of the second cuff piece to latch closed the clip body when the clip body assumes the closed position.

In other embodiments, the adjustable cuff 230 comprises a cuff body 231 comprising a cylindrical surface 232 with a slit 233. The cuff body 231 has grooved edges 234 on either side of the slit 233, wherein the cuff body fits around a catheter lumen and is slidable along the catheter lumen to allow custom positioning of the adjustable cuff along the catheter lumen when the cuff is unlocked. The adjustable cuff 230 further comprises a cuff key 235 comprising projections 236 that fit into the grooved edges 234 at a complementary receiving location for the cuff key, wherein inserting the cuff key into the grooved edges at the receiving location locks the cuff and secures the cuff to the catheter lumen such that the cuff is no longer slidable along the catheter lumen. In some embodiments, the adjustable cuff further comprises a layer of adhesive 237 (e.g., biocompatible silicone) on the inner surface 238 (i.e., lumen-facing surface when attached to a catheter) of the cuff body. In some embodiments, the inner surface of the cuff body further comprises a depression comprising a complementary receiving location for the adhesive. See, e.g., FIGS. 10A-10C and 11A-11B.

In certain embodiments, the modular catheter further comprises a catheter cuff positioning ratchet, wherein the ratchet comprises a plurality of cuff mounting projections that can engage the cuff. In some embodiments, the cuff further comprises a complementary ratchet receiving location designed to mate with at least one cuff mounting projection on the ratchet such that the cuff can be locked in place at the cuff mounting projection. In certain embodiments, the cuff positioning ratchet is attached to the catheter such that the cuff can be positioned along the lumen at a selected cuff mounting projection along the ratchet.

Measurements of the patient can be taken before implantation of the catheter to determine what catheter length will be required between the catheter flow tip and the cuff and the catheter flow tip and the hub. The lumen can be trimmed, for example, on the end that connects to the hub to adjust lumen length. Adding a hub connector and a sealing ring to the trimmed lumen allows the lumen to be connected to the hub by pushing the attached hub connector and the hub together to assemble the catheter. Additionally, a crimp sleeve can be used to secure the catheter to the hub, and the crimp sleeve can be covered with an outer sleeve.

The catheter is positioned within the body of a patient with at least a portion of the hub remaining externally. If the catheter is used for hemodialysis, for example, the lumen is placed in a major vein of the patient, such as an internal jugular vein, a subclavian vein, or a femoral vein. The flow tip of the tunneled catheter is positioned at a target site within the body of a patient. Different placements of the catheter are possible. For example, if the major vein accessed is the right internal jugular vein, the flow tip is positioned in the right atrium. If the major vein accessed is the left internal jugular vein, the flow tip is positioned in the left atrium. If the major vein accessed is the subclavian vein, the flow tip is positioned in the superior vena cava. If the major vein accessed is the femoral vein, the flow tip is positioned in the inferior vena cava. As will be clear to one of skill in the art, other placements of the catheter are possible.

In some embodiments, the catheter is antegrade or retrograde tunneled underneath the skin between a surgical incision site and a target site of interest (e.g., major vein for hemodialysis, abdominal cavity for peritoneal dialysis). For hemodialysis, a portion of the catheter extends through a major vein, a tunneled portion extends under the skin of the patient between the major vein and the incision site (i.e., subcutaneous tunnel), and an exposed portion containing the hub access ports is positioned outside the body. In some embodiments, the catheter further comprises connectors attached to the access ports to connect the catheter with one or more external devices, which may include, without limitation, syringes, pumps, dialyzers, perfusion devices, drainage devices, and the like. The modular catheter may be connected, for example, to a dialysis machine for performing hemodialysis, a cycler machine for performing peritoneal dialysis, or a drainage line to a container for collecting fluid (e.g., plural effusions drained from the chest or malignant ascites drained from the abdomen of the patient).

The lumen may be composed of various polymers including, without limitation, silicone, polyurethane, polyethylene terephthalate, latex, nylon, polyimides, and thermoplastic elastomers. A single lumen or a multi-lumen (e.g., double, or triple lumen) catheter may be used. The use of a multi-lumen catheter allows different intravenous infusions to be connected to each lumen using one catheter access site. For hemodialysis, a double lumen is commonly used, which includes a venous lumen and an arterial lumen that are both placed in the same vein. The arterial lumen (typically colored red) carries blood from the patient to a dialysis machine, whereas the venous lumen (typically colored blue) returns processed blood to the patient from the dialysis machine. In some embodiments, it may be desirable to include an additional lumen, for example, to allow administration of drugs through the catheter into the vein. For peritoneal dialysis, a single lumen is typically used with the lumen tip placed inside the abdominal cavity to allow the intraperitoneal space to be filled with dialysate and drained. The flow tip may have various designs. In some embodiments, the flow tip is a step-tip, split-tip, or symmetrical tip.

The adjustable cuff is positioned on the lumen in the tunneled portion of the catheter. The cuff can be composed of any biocompatible porous material that promotes in-growth from surrounding tissue. Invasion of tissue into the porous material of the cuff secures the cuff so that the catheter cannot be pulled out easily and creates a barrier to prevent spread of infection from the incision site to the inner portions of the body. Tissue in-growth into the cuff also helps to anchor the catheter in the subcutaneous tunnel. The cuff can be made from any suitable biocompatible material that promotes cellular invasion and in-growth. Exemplary materials that can be used in the cuff include, without limitation, Dacron® (polyethylene terephthalate, E. I. DuPont de Nemours, Wilmington, Del.) or other polyesters, polyurethanes, felts, velours, and collagenous materials such as natural, recombinant, or synthetic collagen fibers or harvested extracellular matrix that contains collagen.

In some embodiments, the cuff is further secured to the lumen, for example, using a biocompatible adhesive so as to better adhere the cuff to the lumen and prevent the cuff from moving along the length of the catheter. The adhesive is applied to the surface of the cuff, e.g., before closing the clip body or inserting a key to lock the cuff around the lumen. The adhesive can be applied to the cuff before or after tunneling the catheter under the skin of the patient.

Various types of adhesives may be used to adhere the cuff to the catheter, including, without limitation, medical-grade epoxy, acrylate, cyanoacrylate, silicone, and urethane-based adhesives. In some embodiments an activatable adhesive is used that can be converted from a nonadherent state to an adherent state. For example, activatable adhesives may comprise moisture cured adhesives, enzyme cured adhesives, or multi-component adhesives that require mixing of two or more components that chemically react with one another to cause hardening. In some cases, a chemical reaction may cause polymers to cross link into acrylics, urethanes, and/or epoxies. Multi-component adhesives may include, without limitation, polyester resins mixed with polyurethane resins, polyols mixed with polyurethane resins, and acrylic polymers mixed with polyurethane resins. Individually, the components of multi-component adhesives are typically not adhesive by nature but demonstrate adhesion after being mixed together and cured.

In some embodiments, an activatable adhesive comprises a reactive-type adhesive that hardens via a chemical reaction with an external energy source such as ultraviolet (UV) or visible light and/or heat. When the external energy source is applied to the reactive-type adhesive a chemical reaction occurs, and the activatable adhesive adheres the cuff to the catheter.

In some embodiments, a non-reactive adhesive is used, such as an adhesive that hardens by drying, application of pressure, contact, and/or heat. An example of a pressure-sensitive adhesive is medical tape, which adheres by applying pressure to couple the adhesive with the surface of the body being adhered. Pressure sensitive adhesives may be either permanent or removable-type adhesives and can be manufactured in a liquid or solid form. In some embodiments, hot adhesives are used that require the heating, liquefaction, and subsequent solidification of thermal plastics. An example of a hot adhesive is ethylene-vinyl acetate, which can be melted into a liquid form and then solidified to adhere components together.

Catheters can be implanted into patients by using surgical techniques well-known in the art, including surgical laparoscopy or laparotomy. Exemplary methods include without limitation the Seldinger technique, the modified Seldinger technique, the Trocar technique, and the Moncrief-Popovich technique. Additionally, tunneled catheters may be implanted using ultrasound guidance to identify the target site (e.g., vein for hemodialysis or abdominal cavity for peritoneal dialysis) and guide puncture and catheter placement.

### Systems Comprising a Modular Catheter

In various embodiments, systems are provided comprising a modular catheter, as described herein, connected to one or more external devices, which may include, without limitation, syringes, pumps, dialyzers, perfusion devices, drainage devices, and the like. The modular catheter may be connected, for example, to a dialysis machine for performing hemodialysis, a cycler machine for performing peritoneal dialysis, or a drainage line to a container for collecting fluid.

In certain embodiments, a hemodialysis system comprising a modular catheter and a dialysis machine is provided. The hemodialysis system may comprise the modular catheter together with, e.g., a dialyzer, a blood pressure monitor, and a pump. For hemodialysis, the modular catheter is customized to the patient by trimming the lumen to optimize the cuff-to-tip and/or tip-to-hub distances for a selected placement of the catheter in a major vein based on the patient's measurements. The trimmed lumen is connected to the hub through the hub connector, and the modular catheter is implanted in a subcutaneous tunnel with the lumen placed within a major vein (e.g., right internal jugular vein with the flow tip in the right atrium, left internal jugular vein with the flow tip in the left atrium, subclavian vein with the flow tip in the superior vena cava, or femoral vein with the flow tip in the inferior vena cava) in the patient. A portion of the modular catheter may be antegrade or retrograde tunneled beneath the skin. The adjustable cuff is placed on the lumen at a selected position in the tunneled portion of the catheter. To begin dialysis, the hub access ports are connected to the dialysis machine. One hub access port is used for blood leaving the patient's body, and the other hub access port is used for blood returning to the patient's body after being processed by the dialysis machine.

In another embodiment, a peritoneal dialysis system comprising a modular catheter and a cycler machine is provided. For peritoneal dialysis, the modular catheter is customized to the patient by trimming the lumen to optimize the cuff-to-tip and/or tip-to-hub distances for catheter implantation in the patient's abdomen. The modular catheter is implanted with the lumen flow tip within the abdominal cavity. A portion of the modular catheter may be antegrade or retrograde tunneled beneath the skin. The adjustable cuff is placed on the lumen at a selected position in the tunneled portion of the catheter. To begin peritoneal dialysis, the hub access ports are connected to a cycler machine. Repeated cycles of filling the abdomen with dialysate followed by draining the dialysate fluid from the abdomen are performed.

In another embodiment, a fluid drainage system comprising a modular catheter and a drainage container for collecting fluid is provided. The drainage system can be used for draining fluid, for example, from the lungs abdomen, liver, abscesses, cysts, pseudocysts, pneumothoraces, hematomas, bilomas, or urinomas. For example, the fluid drainage system can be used for draining plural effusions from the chest, or malignant ascites from the abdomen of a patient. The lumen length and flow tip spacing are customized based on measurements of the patient taken at the site in need of fluid drainage. The modular catheter is implanted with the lumen flow tip within the site of interest to allow fluid drainage. A portion of the modular catheter may be antegrade or retrograde tunneled beneath the skin. The adjustable cuff is placed on the lumen at a selected position in the tunneled portion of the catheter. To begin fluid drainage, the hub access ports are connected to an external drainage line on a drainage container, and the fluid is collected in the drainage container.

### Kits

Also provided are kits comprising an adjustable cuff or a modular catheter as described herein. In some embodiments, the assembled catheter is contained in a sterile package. Alternatively, the kit may contain the unassembled components of the catheter, including the hub, the trimmable lumen, and the adjustable cuff contained in one or more sterile packages.

In addition to the above components, the subject kits may further include (in certain embodiments) instructions for practicing the subject methods. In some embodiments, instructions for using the modular catheter for hemodialysis, peritoneal dialysis, and/or draining fluid from a patient are provided in the kits. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-52 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. An adjustable cuff comprising:
a) a cuff body comprising a cylindrical surface with a slit, said cuff body having grooved edges on either side of the slit, wherein the cuff body fits around a catheter lumen and is slidable along the catheter lumen to allow custom positioning of the adjustable cuff along the catheter lumen when the cuff is unlocked; and
b) a cuff key comprising projections that fit into the grooved edges at a complementary receiving location for the cuff key, wherein inserting the cuff key into the grooved edges at the receiving location locks the cuff and secures the cuff to the catheter lumen such that the cuff is no longer slidable along the catheter lumen.

2. The adjustable cuff of aspect 1, further comprising a layer of adhesive on the inner surface of the cuff body.

3. The adjustable cuff of aspect 2, wherein the inner surface of the cuff body further comprises a depression comprising a complementary receiving location for the adhesive.

4. The adjustable cuff of aspect 3, wherein the adhesive is a biocompatible silicone.

5. An adjustable cuff comprising a clip body that can assume an open position or a closed position, said clip body further comprising a cuff material on the surface of the clip body with an adhesive-compatible surface, wherein the clip body comprises a first cuff piece and a second cuff piece, each cuff piece connected to a central hinge, wherein the first cuff piece and the second cuff piece can snap together around a catheter lumen to allow custom positioning of the adjustable cuff along the catheter lumen.

6. The adjustable cuff of aspect 5, wherein the clip body further comprises an external latching projection on the first cuff piece and a complementary latch receiving location on the second cuff piece, wherein the external latching projection of the first cuff piece fits inside the receiving location of the second cuff piece to latch closed the clip body when the clip body assumes the closed position.

7. The adjustable cuff of aspect 5 or 6, wherein the clip body further comprises an adhesive on the adhesive-compatible surface of the cuff material.

8. The adjustable cuff of aspect 7, wherein the adhesive is an activatable adhesive.

9. The adjustable cuff of any one of aspects 1 to 8, wherein the cuff material comprises polyethylene terephthalate, a polyester, a polyurethane, a felt, a velour, collagen fibers, or an extracellular matrix.

10. A modular catheter comprising:
a) a hub comprising a pair of access ports, wherein the pair of access ports comprises a first access port for a fluid exiting a body of a patient and a second access port for a fluid entering the body of the patient;
b) a trimmable lumen that can be trimmed to a customizable length, wherein the trimmable lumen comprises: i) a first end that is trimmable, wherein the first end is attached to a hub connector and a sealing ring that are used to connect the first end of the trimmable lumen to the hub by push-together assembly, and ii) a second end comprising a flow tip comprising a plurality of venous inlets and arterial outlets; and
c) the adjustable cuff of any one of aspects 1 to 9.

11. The modular catheter of aspect 10, wherein the adjustable cuff is positioned on the trimmable lumen such that the length of a first section of the trimmable lumen between the flow tip and the modular venous-arterial hub and the length of a second section of the trimmable lumen between the flow tip and the adjustable cuff are customized to optimize tip spacing for a patient.

12. The modular catheter of aspect 11, wherein the length of the lumen and the position of the adjustable cuff are customized for antegrade tunneling or retrograde tunneling.

13. The modular catheter of aspect 11 or 12, wherein the length of the lumen and the position of the adjustable cuff are customized to allow placement of the lumen in a major vein of the patient.

14. The modular catheter of aspect 13, wherein the major vein is selected from the group consisting of an internal jugular vein, a subclavian vein, and a femoral vein.

15. The modular catheter of aspect 14, wherein i) the major vein is the right internal jugular vein, and the length of the lumen and the position of the adjustable cuff are customized to allow placement of the flow tip in the right atrium; ii) the major vein is the left internal jugular vein, and the length of the lumen and the position of the adjustable cuff are customized to allow placement of the flow tip in the left atrium; iii) the major vein is the subclavian vein, and the length of the lumen and the position of the adjustable cuff are customized to allow placement of the flow tip in the superior vena cava; or iv) the major vein is the femoral vein, and the length of the lumen and the position of the adjustable cuff are customized to allow placement of the flow tip in the inferior vena cava.

16. The modular catheter of any one of aspects 10 to 15, wherein the catheter is sized for a pediatric patient.

17. The modular catheter of aspect 16, wherein the pediatric patient is an adolescent or an infant.

18. The modular catheter of any one of aspects 10 to 17, wherein the trimmable lumen comprises polyurethane or silicone.

19. The modular catheter of any one of aspects 10 to 18, wherein the trimmable lumen is a single lumen, a double lumen, or a triple lumen.

20. The modular catheter of any one of aspects 10 to 19, wherein the length of the modular catheter ranges from about 2 cm to about 22 cm.

21. The modular catheter of aspect 20, wherein the length of the modular catheter ranges from about 2 cm to about 12 cm.

22. The modular catheter of any one of aspects 10 to 21, wherein the trimmable lumen has a French size ranging from 3 french to 34 french.

23. The modular catheter of aspect 22, wherein the trimmable lumen has a French size ranging from 10 french to 16 french.

24. The modular catheter of aspect 23, wherein the trimmable lumen has a French size ranging from 3 french to 8 french.

25. The modular catheter of any one of aspects 10 to 24, further comprising a catheter cuff positioning ratchet, wherein the ratchet comprises a plurality of cuff mounting projections that can engage the cuff, wherein the cuff positioning ratchet is attached to the catheter such that the cuff can be positioned along the lumen at a selected cuff mounting projection along the ratchet.

26. The modular catheter of aspect 25, wherein the cuff further comprises a complementary ratchet receiving location designed to mate with at least one cuff mounting projection on the ratchet such that the cuff can be locked in place at the cuff mounting projection.

27. The modular catheter of any one of aspects 10 to 26, wherein the flow tip is a step-tip, split-tip, or symmetrical tip.

28. A hemodialysis system comprising the modular catheter of any one of aspects 10 to 27 and a dialyzer.

29. The hemodialysis system of aspect 28, further comprising a blood pressure monitor.

30. The hemodialysis system of aspect 28 or 29, further comprising a pump.

31. The hemodialysis system of any one of aspects 28 to 30, further comprising a dialysis machine.

32. A peritoneal dialysis system comprising the modular catheter of any one of aspects 10 to 27 and a cycler machine.

33. A fluid drainage system comprising the modular catheter of any one of aspects 10 to 27 and a drainage container for collecting fluid.

34. The fluid drainage system of aspect 33, wherein the fluid comprises plural effusions drained from the chest or malignant ascites drained from the abdomen of a patient.

35. A method of using the modular catheter of any one of aspects 10 to 27 to perform hemodialysis on a patient, the method comprising:
a) taking measurements of the patient at a site of interest for catheter implantation;
b) trimming the first end of the trimmable lumen and positioning and closing the adjustable cuff on the trimmable lumen of the modular catheter of any one of aspects 10 to 27 to customize the lumen length and flow tip spacing according to the measurements of the patient;
c) attaching the hub connector and the sealing ring to the first end of the trimmable lumen;
d) connecting the hub connector to the hub;
e) implanting the modular catheter into a subcutaneous tunnel at the site of interest;
f) placing the trimmable lumen within a major vein of the patient;
g) positioning the flow tip in a suitable location based on said placing of the trimmable lumen within the major vein;
h) connecting the pair of access ports of the hub to a dialysis machine, wherein the first access port is used for blood leaving the patient's body and the second access port is used for blood returning to the patient's body after being processed by the dialysis machine; and
i) performing the hemodialysis on the patient.

36. The method of aspect 35, wherein the major vein is selected from the group consisting of an internal jugular vein, a subclavian vein, and a femoral vein.

37. The method of aspect 36, wherein i) the major vein is the right internal jugular vein, and the flow tip is positioned in the right atrium; ii) the major vein is the left internal jugular vein, and the flow tip is positioned in the left atrium; iii) the major vein is the subclavian vein, and the flow tip is positioned in the superior vena cava; or iv) the major vein is the femoral vein, and the flow tip is positioned in the inferior vena cava.

38. The method of any one of aspects 35 to 37, further comprising: tunneling a portion of the modular catheter beneath the skin of the patient.

39. The method of aspect 38, wherein said tunneling is antegrade tunneling or retrograde tunneling.

40. The method of any one of aspects 35 to 39, further comprising monitoring blood pressure of the patient while said performing hemodialysis.

41. A method of using the modular catheter of any one of aspects 10 to 27 to perform peritoneal dialysis on a patient, the method comprising:
a) taking measurements of the patient at a site of interest for catheter implantation in the patient's abdomen;
b) trimming the first end of the trimmable lumen and positioning and closing the adjustable cuff on the trimmable lumen of the modular catheter of any one of aspects 10 to 27 to customize the lumen length and flow tip spacing according to the measurements of the patient;
c) attaching the hub connector and the sealing ring to the first end of the trimmable lumen;
d) connecting the hub connector to the hub;
e) implanting the modular catheter at the site of interest in the abdomen;
f) placing the trimmable lumen within a subcutaneous tunnel at the site of interest in the abdomen of the patient;
g) positioning the lumen flow tip within the abdominal cavity;
h) connecting the pair of access ports of the hub to a cycler machine, wherein the first access port is used for draining fluid from the abdomen and the second access port is used for filling the abdomen with dialysate; and
i) performing the peritoneal dialysis on the patient.

42. The method of aspect 41, further comprising: tunneling a portion of the modular catheter beneath the skin of the patient.

43. The method of aspect 42, wherein said tunneling is antegrade tunneling or retrograde tunneling.

44. A method of using the modular catheter of any one of aspects 10 to 27 to drain fluid from a patient, the method comprising:
a) taking measurements of the patient at site of interest for catheter implantation to allow fluid drainage;
b) trimming the first end of the trimmable lumen and positioning and closing the adjustable cuff on the trimmable lumen of the modular catheter of any one of aspects 10 to 27 to customize the lumen length and flow tip spacing according to the measurements of the patient;
c) attaching the hub connector and the sealing ring to the first end of the trimmable lumen;
d) connecting the hub connector to the hub;
e) implanting the modular catheter at the site of interest;
f) placing the trimmable lumen within a subcutaneous tunnel at the site of interest in the patient;
g) positioning the flow tip within the site of interest to allow fluid drainage;
h) connecting at least one access port of the hub to a drainage line on a drainage container;
i) collecting the fluid in the drainage container.

45. The method of aspect 44, wherein the site of interest for catheter implantation is in the chest or abdomen of the patient.

46. The method of aspect 45, wherein the fluid comprises plural effusions drained from the chest or malignant ascites drained from the abdomen of the patient.

47. The method of any one of aspects 44 to 46, further comprising: tunneling a portion of the modular catheter beneath the skin of the patient.

48. The method of aspect 47, wherein said tunneling is antegrade tunneling or retrograde tunneling.

49. The method of any one of aspects 35 to 48, further comprising suturing the modular catheter securely in place in the patient.

50. The method of any one of aspects 35 to 49, further comprising administering an anti-coagulant, an antibiotic, or an analgesic agent to the patient.

51. A kit comprising the cuff of any one of aspects 1 to 9 or the modular catheter of any one of aspects 10 to 27 and instructions for using the cuff or the modular catheter for hemodialysis, peritoneal dialysis, or draining fluid from a patient.

52. The kit of aspect 51, wherein the modular catheter is assembled and contained in a sterile package, or unassembled with the hub, the trimmable lumen, and the adjustable cuff contained in one or more sterile packages.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### A Pediatric Modular Catheter System

Pediatric patients require hemodialysis for end stage renal disease (ESRD) and acute kidney injury. In children, the optimal route of renal clearance is peritoneal dialysis, however, many children require hemodialysis either as a temporary means of renal clearance (i.e. 26.9% of the children in the ICU develop acute kidney injury (AKI) and of these 6% require dialysis), or as a more long-term solution while awaiting a renal transplant. Very few commercially available hemodialysis catheters are specifically designed for children.

The current size of hemodialysis catheters (both diameter=French (Fr) size and length) is based on the average sized adult patient. However, pediatric patients typically range from about 10 kg to 70 kg. Children with chronic kidney failure are also more likely to be small in stature, given their chronic disease, compounding the mismatch with adult average size catheters. Problems with using catheters, designed for adults, on children include: (i) the adult catheters have a suboptimal length for the subcutaneous tunnel and for the internal intravenous portion of the catheter, (ii) the external diameter (Fr) of the catheter is too large relative to the small vein size of children and (iii) problems with flow rates given limitations related to the number and configuration of side-holes and catheter tip design, especially due to the small size of the superior vena cava and right atrium. In addition, the very short internal intravenous catheter length needed for most small children leads to an increased risk of blood loss and the potential for air embolism with current commercially available catheters.

Here we describe a pediatric modular catheter system better suited to the needs of children than the adult catheter systems currently available. This system is designed with customized lengths and a better port location that takes into account the smaller size of children. This system offers improved usability and greater comfort to pediatric patients with fewer complications.

We have developed a 6 Fr dual lumen polyurethane dialysis catheter which can be placed over a wire. This catheter can be cut (i.e. has a modular length) so it can be tunneled to any location on the body of a pediatric patient, regardless of size. A 0.5 cm cuff has been designed that is “clipped” externally around the catheter such that it can be positioned subcutaneously at the exit dermatotomy site for catheter securement (FIG. 1). Once the catheter is cut, it is then attached to a hub or connector (FIG. 2).

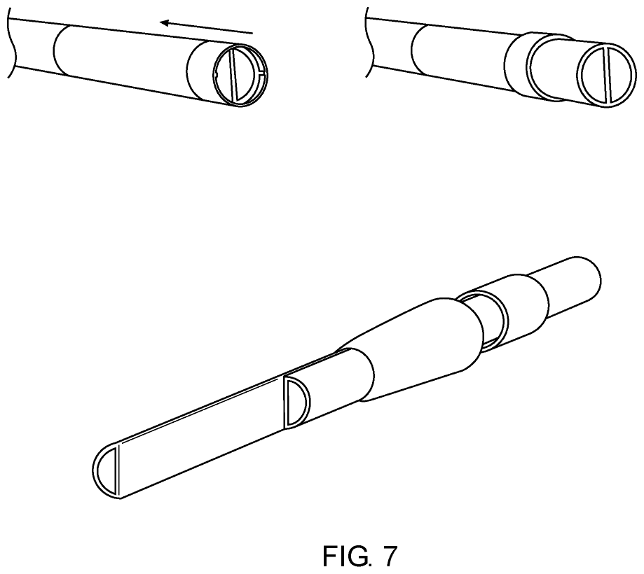

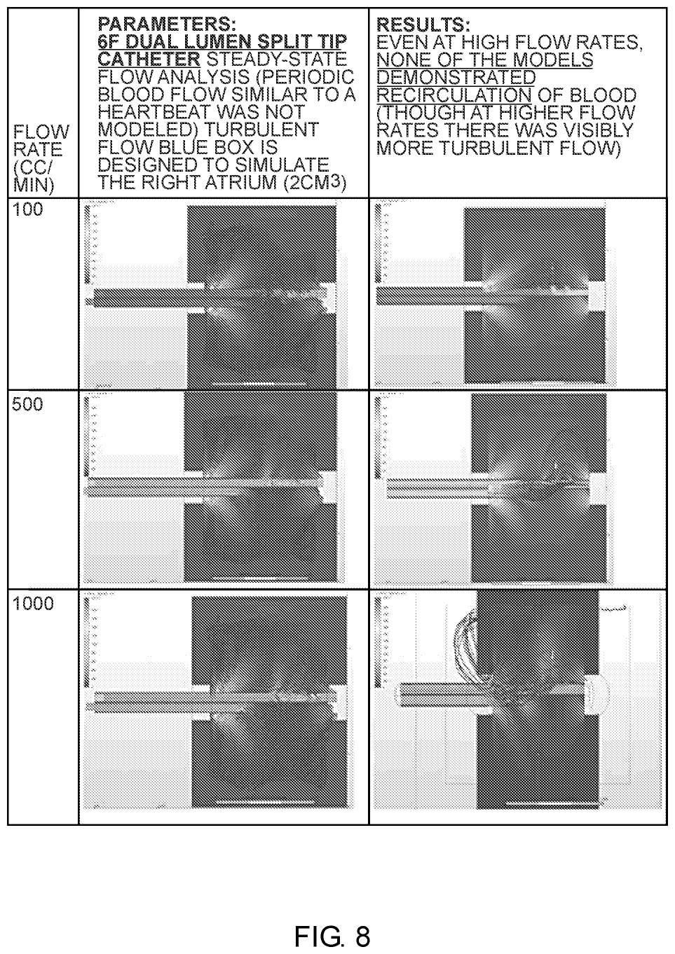

We have also designed a short split-tip catheter (which has holes only 3 cm at the catheter tip—CDF modeling has shown no blood re-circulation with this design; FIG. 8). We are also designing a single-tip catheter which we expect will be better suited to pediatric patients. The hole design (size, number and configuration) should be optimized to ensure no re-circulation for efficient dialysis at the required rates (i.e. >100 cc/min).

### Example 2

### Pediatric Modular Catheter Prototypes

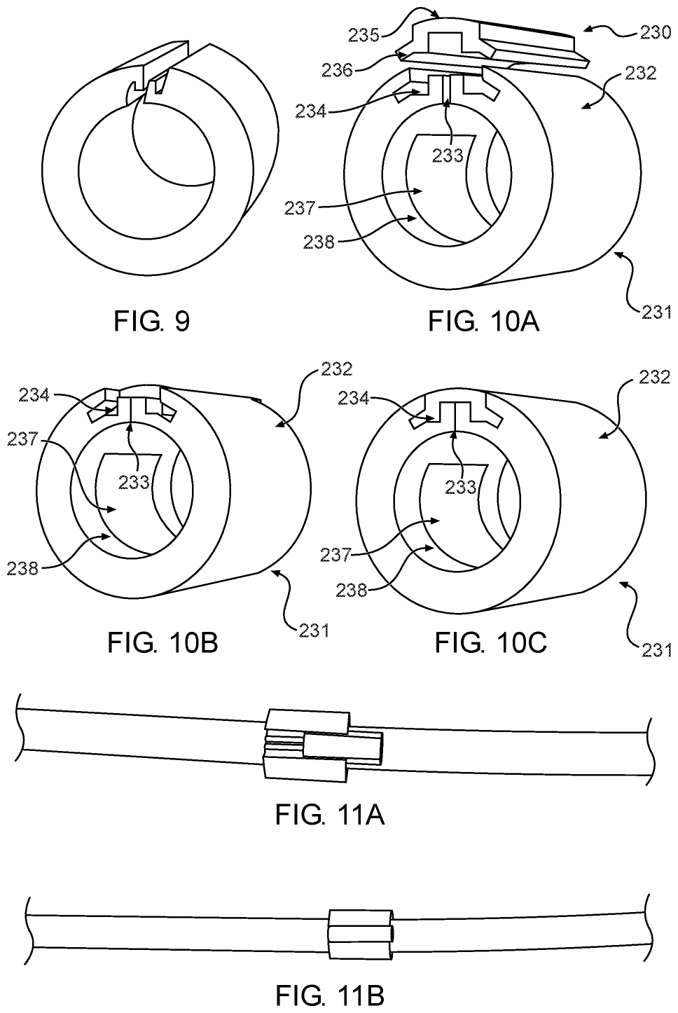

We prototyped multiple designs of the adjustable catheter cuff, focusing on iterations of the locking mechanism to secure the cuff on the catheter once properly positioned. Initial prototypes were 3D-printed from a rigid biocompatible polyurethane (MPU100, Carbon, Redwood City, Calif.) and were designed as a single body with a slit along the longitudinal axis that enabled smooth motion of the cuff along the catheter (see FIG. 9). The edges of the slit were designed such that they acted as a clamp: when the cuff was squeezed, the clamp engaged, and the cuff became a seamless cylinder secured to the catheter with significant holding force. However, due to the low profile necessary for the catheter cuff (outer diameter <8 mm; inner diameter=5 mm), and the resulting small features of this clamping mechanism, this design was not as robust as desired for repositioning of the cuff with repeated clamping and un-clamping.

A second set of prototypes was developed to improve robustness for re-positioning. This design is comprised of two parts: a main body with a longitudinal slit featuring grooves, and a smaller key with features that slot into the grooves (see FIGS. 10A-10C). When the key is slotted into the main cuff body, the cuff clamps down on the catheter, securing it in position. The main body additionally includes an inner layer of biocompatible silicone (SIL30, Carbon) that is 3D-printed to precisely fit into a matching depression on the inner face of the cuff main body. This silicone component significantly increases the friction between the catheter and cuff when clamped, thus resulting in a higher holding force (see FIGS. 11A, 11B).

1. An adjustable cuff comprising:
a) a cuff body comprising a cylindrical surface with a slit, said cuff body having grooved edges on either side of the slit, wherein the cuff body fits around a catheter lumen and is slidable along the catheter lumen to allow custom positioning of the adjustable cuff along the catheter lumen when the cuff is unlocked; and
b) a cuff key comprising projections that fit into the grooved edges at a complementary receiving location for the cuff key, wherein inserting the cuff key into the grooved edges at the receiving location locks the cuff and secures the cuff to the catheter lumen such that the cuff is no longer slidable along the catheter lumen.

2. The adjustable cuff of claim 1, further comprising a layer of adhesive on the inner surface of the cuff body.

3. The adjustable cuff of claim 2, wherein the inner surface of the cuff body further comprises a depression comprising a complementary receiving location for the adhesive.

4. The adjustable cuff of claim 3, wherein the adhesive is a biocompatible silicone adhesive.

5. An adjustable cuff comprising a clip body that can assume an open position or a closed position, said clip body further comprising a cuff material on the surface of the clip body with an adhesive-compatible surface, wherein the clip body comprises a first cuff piece and a second cuff piece, each cuff piece connected to a central hinge, wherein the first cuff piece and the second cuff piece can snap together around a catheter lumen to allow custom positioning of the adjustable cuff along the catheter lumen.

6. The adjustable cuff of claim 5, wherein the clip body further comprises an external latching projection on the first cuff piece and a complementary latch receiving location on the second cuff piece, wherein the external latching projection of the first cuff piece fits inside the receiving location of the second cuff piece to latch closed the clip body when the clip body assumes the closed position.

7-9. (canceled)

10. A modular catheter comprising:
a) a hub comprising a pair of access ports, wherein the pair of access ports comprises a first access port for a fluid exiting a body of a patient and a second access port for a fluid entering the body of the patient;
b) a trimmable lumen that can be trimmed to a customizable length, wherein the trimmable lumen comprises: i) a first end that is trimmable, wherein the first end is attached to a hub connector and a sealing ring that are used to connect the first end of the trimmable lumen to the hub by push-together assembly, and ii) a second end comprising a flow tip comprising a plurality of venous inlets and arterial outlets; and
c) the adjustable cuff of claim 1.

11. The modular catheter of claim 10, wherein the adjustable cuff is positioned on the trimmable lumen such that the length of a first section of the trimmable lumen between the flow tip and the modular venous-arterial hub and the length of a second section of the trimmable lumen between the flow tip and the adjustable cuff are customized to optimize tip spacing for a patient.

12. The modular catheter of claim 11, wherein the length of the lumen and the position of the adjustable cuff are customized for antegrade tunneling or retrograde tunneling.

13. The modular catheter of claim 11, wherein the length of the lumen and the position of the adjustable cuff are customized to allow placement of the lumen in a major vein of the patient.

14-15. (canceled)

16. The modular catheter of claim 10, wherein the catheter is sized for a pediatric patient.

17. (canceled)

18. The modular catheter of claim 10, wherein the catheter comprises polyurethane or silicone.

19. The modular catheter of claim 10, wherein the trimmable lumen is a single lumen, a double lumen, or a triple lumen.

20. The modular catheter of claim 10, wherein the length of the modular catheter ranges from about 2 cm to about 22 cm, and the lumen has a French size ranging from 3 french to 34 french.

21-24. (canceled)

25. The modular catheter of claim 10, further comprising a catheter cuff positioning ratchet, wherein the ratchet comprises a plurality of cuff mounting projections that can engage the cuff, wherein the cuff positioning ratchet is attached to the catheter such that the cuff can be positioned along the lumen at a selected cuff mounting projection along the ratchet, wherein the cuff further comprises a complementary ratchet receiving location designed to mate with at least one cuff mounting projection on the ratchet such that the cuff can be locked in place at the cuff mounting projection.

26. (canceled)

27. The modular catheter of claim 10, wherein the flow tip is a step-tip, split-tip, or symmetrical tip.

28. A hemodialysis system comprising the modular catheter of claim 10 and a dialyzer.

29-31. (canceled)

32. A peritoneal dialysis system comprising the modular catheter of claim 10 and a cycler machine.

33. A fluid drainage system comprising the modular catheter of claim 10 and a drainage container for collecting fluid.

34. (canceled)

35. A method of using the modular catheter of claim 10 to perform hemodialysis on a patient, the method comprising:
a) taking measurements of the patient at a site of interest for catheter implantation;
b) trimming the first end of the trimmable lumen and positioning and closing the adjustable cuff at a selected position on the trimmable lumen of the modular catheter of claim 10 to customize the lumen length and flow tip spacing according to the measurements of the patient;
c) attaching the hub connector and the sealing ring to the first end of the trimmable lumen;
d) connecting the hub connector to the hub;
e) implanting the modular catheter into a subcutaneous tunnel at the site of interest;
f) placing the trimmable lumen within a major vein of the patient;
g) positioning the flow tip in a suitable location based on said placing of the trimmable lumen within the major vein;
h) connecting the pair of access ports of the hub to a dialysis machine, wherein the first access port is used for blood leaving the patient's body and the second access port is used for blood returning to the patient's body after being processed by the dialysis machine; and
i) performing the hemodialysis on the patient.

36-40. (canceled)

41. A method of using the modular catheter of claim 10 to perform peritoneal dialysis on a patient, the method comprising:
a) taking measurements of the patient at a site of interest for catheter implantation in the patient's abdomen;
b) trimming the first end of the trimmable lumen and positioning and closing the adjustable cuff at a selected position on the trimmable lumen of the modular catheter of claim 10 to customize the lumen length and flow tip spacing according to the measurements of the patient;
c) attaching the hub connector and the sealing ring to the first end of the trimmable lumen;
d) connecting the hub connector to the hub;
e) implanting the modular catheter at the site of interest in the abdomen;
f) placing the trimmable lumen within a subcutaneous tunnel at the site of interest in the abdomen of the patient;
g) positioning the lumen flow tip within the abdominal cavity;
h) connecting the pair of access ports of the hub to a cycler machine, wherein the first access port is used for draining fluid from the abdomen and the second access port is used for filling the abdomen with dialysate; and
i) performing the peritoneal dialysis on the patient.

42-43. (canceled)

44. A method of using the modular catheter of claim 10 to drain fluid from a patient, the method comprising:
a) taking measurements of the patient at site of interest for catheter implantation to allow fluid drainage;
b) trimming the first end of the trimmable lumen and positioning and closing the adjustable cuff at a selected position on the trimmable lumen of the modular catheter of claim 10 7 to customize the lumen length and flow tip spacing according to the measurements of the patient;
c) attaching the hub connector and the sealing ring to the first end of the trimmable lumen;
d) connecting the hub connector to the hub;
e) implanting the modular catheter at the site of interest;
f) placing the trimmable lumen within a subcutaneous tunnel at the site of interest in the patient;
g) positioning the flow tip within the site of interest to allow fluid drainage;
h) connecting at least one access port of the hub to a drainage line on a drainage container;
i) collecting the fluid in the drainage container.

45-52. (canceled)

53. A modular catheter comprising:
a) a hub comprising a pair of access ports, wherein the pair of access ports comprises a first access port for a fluid exiting a body of a patient and a second access port for a fluid entering the body of the patient;
b) a trimmable lumen that can be trimmed to a customizable length, wherein the trimmable lumen comprises: i) a first end that is trimmable, wherein the first end is attached to a hub connector and a sealing ring that are used to connect the first end of the trimmable lumen to the hub by push-together assembly, and ii) a second end comprising a flow tip comprising a plurality of venous inlets and arterial outlets; and
c) the adjustable cuff of claim 5.
